(19) United States
(12) Patent Application Publication
(10) Pub. No.: US 2022/0175749 A1
(43) Pub. Date: Jun. 9, 2022

(54) LANABECESTAT FOR WEIGHT LOSS

(71) Applicant: ASTRAZENECA AB, Södertälje (SE)

(72) Inventors: Craig SHERING, Waltham, MA (US); John SIMS, Indianapolis, IN (US)

(21) Appl. No.: 17/437,934

(22) PCT Filed: Mar. 13, 2020
(86) PCT No.: PCT/EP2020/056994

§ 371 Date: Sep. 10, 2021

Related U.S. Application Data
(60) Provisional application No. 62/818,718, filed on Mar. 14, 2019.

(51) Int. Cl. A61K 31/4439; A61P 3/04; A61K 45/06

(52) U.S. Cl. CPC A61K 31/4439; A61K 45/06; A61P 3/04

(57) ABSTRACT

The present application relates to affecting weight loss comprising administering a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (e.g., the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine).

### RELATED APPLICATION

This application claims the benefit of priority to U.S. Provisional Patent Application No. 62/818,718, filed Mar. 14, 2019, which application is hereby incorporated by reference in its entirety.

### BACKGROUND

Obesity is one of the leading preventable cause of death worldwide. In 2015, 600 million adults (12%) and 100 million children were obese in 195 countries (“Health Effects of Overweight and Obesity in 195 Countries over 25 Years.” The New England Journal of Medicine. 377 (1): 13-27), with experts viewing obesity as one of the most serious public health problems of the 21st century (“Role of the gastroenterologist in managing obesity.” Expert Review of Gastroenterology & Hepatology (Review). 7 (5): 439-51). Indeed, the likeliehood of numerous additional conditions, such as cardiovascular diseases, type 2 diabetes, sleep disorders, certain cancers, osteoarthritis, and mood disorders, are increased as a result of obesity.

There is presently no effective pharmaceutical intervention for the prevention of weight gain or for prevention or treatment of obesity. While traditional pharmacotherapies for treating overweight or obese subjects may provide short-term benefits, they are often associated with relapse and potentially harmful side effects. Accordingly, there is a need for safe and effective thereapeutic interventions.

### SUMMARY OF APPLICATION

The present application provides a method of affecting weight loss in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

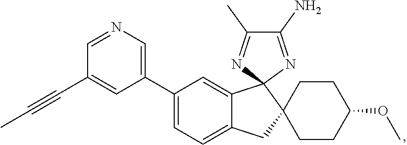 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof. In certain embodiments, the weight of the subject decreases by at least 5%, at least 6%, at least 7%, at least 8%, at least 9%, at least 10%, at least 15%, at least 20%, or at least 30%. In certain embodiments, the weight loss is maintained for at least one month. In certain embodiments, the weight loss is maintained for at least one year.

The present application provides a method of preventing weight gain in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

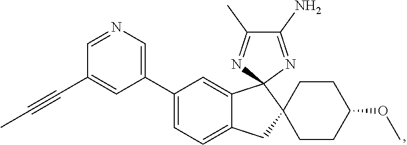 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof. In certain embodiments, the weight of the subject increases by less than 5%. In certain embodiments, the prevention of weight gain is maintained for at least one month. In certain embodiments, the prevention of weight gain is maintained for at least one year.

The present application provides a method of reducing body mass index (BMI) in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

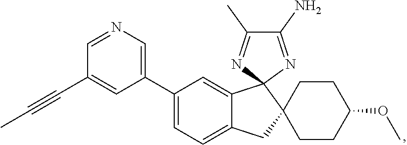 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof. In certain embodiments, the subject has a BMI of at least about 25 kg/m2 prior to the administration. In certain embodiments, the BMI is reduced by at least 5%, at least 6%, at least 7%, at least 8%, at least 9%, at least 10%, at least 15%, at least 20%, or at least 30%. In certain embodiments, the subject has a BMI of less than 25 kg/m2 after the administration of the therapeutically effective amount. In certain embodiments of the foregoing, the reduced BMI is maintained for at least one month. In certain embodiments of the foregoing, the reduced BMI is maintained for at least one year.

The present application provides a method of treating a condition or disorder associated with being overweight or obese in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

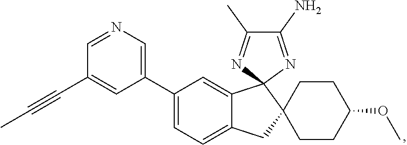 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof. In certain embodiments, the condition or disorder associated with being overweight or obese comprises a cardiac disorder, an endocrine disorder, a respiratory disorder, a hepatic disorder, a skeletal, a psychiatric disorder, a metabolic disorder, a sleeping disorder, a reproductive disorder, or a combination thereof.

In certain embodiments of any of the foregoing methods, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

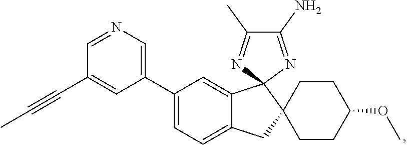 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof, is conjointly administered with a second weight loss agent. In certain such embodiments, the second weight loss agent is selected from serotonin and noradrenergic re-uptake inhibitors, noradrenergic re-uptake inhibitors, selective serotonin re-uptake inhibitors, intestinal lipase inhibitors, orlistat, sibutramine, methamphetamine, ionamin, phentermine, bupropion, diethylpropion, phendimetrazine, benzphetermine, pramlintide, exenatide, liraglutide, and Topamax.

In certain embodiments of any of the foregoing methods, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered orally.

In certain embodiments of any of the foregoing methods, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered once daily.

In certain embodiments of any of the foregoing methods, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered at a dose of 20 mg/day.

In certain embodiments of any of the foregoing methods, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered at a dose of 50 mg/day.

In certain embodiments of any of the foregoing methods, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered once daily for at least 5 weeks. In certain such embodiments, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered once daily for at least 25 weeks. In yet further embodiments, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered for at least 50 weeks.

In certain embodiments of any of the foregoing methods, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

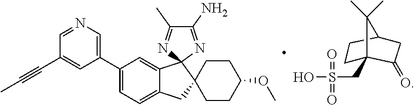 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

The present application provides use of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, in the preparation of a medicament for affecting weight loss.

### BRIEF DESCRIPTION OF THE DRAWINGS

FIG. 1 depicts the weight change from baseline over the course of the 104 week study described in Example 3 for the overall population.

FIG. 2 depicts the weight change from baseline over the course of a 104 week study described in Example 3 for the population with a baseline BMI≤25 kg/m².

FIG. 3 depicts the weight change from baseline over the course of a 104 week study described in Example 3 for the population with a baseline BMI>25 kg/m².

### DETAILED DESCRIPTION OF THE APPLICATION

The present application provides methods of affecting weight loss in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

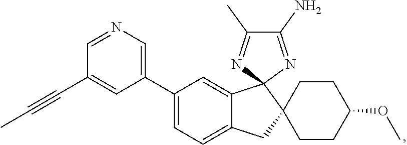 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

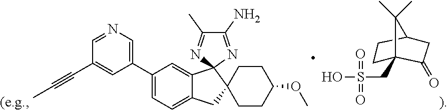 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

In certain embodiments, the weight of the subject decreases by at least 5%, at least 6%, at least 7%, at least 8%, at least 9%, at least 10%, at least 15%, at least 20%, at least 25%, or at least 30%. In certain such embodiments, the weight of the subject decreases by at least 7%. In certain such embodiments, the weight of the subject decreases by at least 5%. In certain such embodiments, the weight of the subject decreases by at least 10%. In certain embodiments, the method further comprises maintaining the weight loss for at least one month, at least two months, at least three months, at least four months, at least five months, or at least six months. In certain embodiments, the method further comprises maintaining the weight loss for at least one year, such as at least two years. In certain embodiments of the foregoing, the subject has a baseline BMI (i.e., has a BMI at the start of treatment) of greater than 25 kg/m².

The present application provides methods of preventing weight gain comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

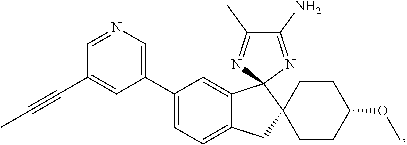 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

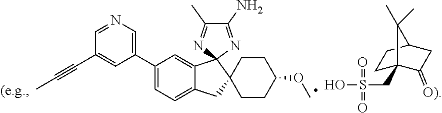

In certain embodiments, the weight of the subject increases by less than 7%, or less than 5%. In certain such embodiments, the method further comprises maintaining the weight increase of less than 7%, or less than 5%, for at least one month, at least two months, at least three months, at least four months, at least five months, or at least six months. In certain such embodiments, the method further comprises maintaining the weight increase of less than 7%, or less than 5%, for at least one year, such as at least two years. In certain embodiments of the foregoing, the subject has a baseline BMI (i.e., has a BMI at the start of treatment) of greater than 25 kg/m².

The present application provides methods of suppressing appetite comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

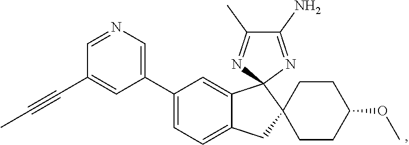 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

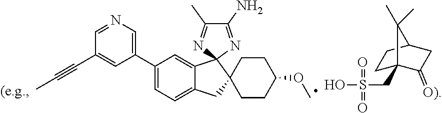

The present application provides methods of treating obesity comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

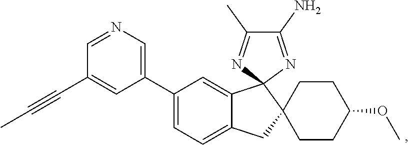 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

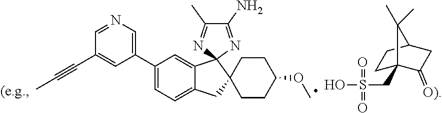

The present application provides methods of reducing body mass index (BMI) in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

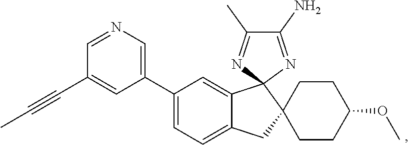 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

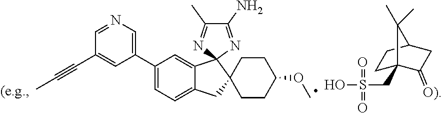

In certain such embodiments, the method comprises reducing the BMI of the subject by at least 5%, at least 6%, at least 7%, at least 8%, at least 9%, at least 10%, at least 15%, at least 20%, at least 25%, or at least 30%. In certain embodiments of the foregoing method, the subject has a BMI of at least about 25 kg/m2, or at least about 30 kg/m2 prior to administration of the therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

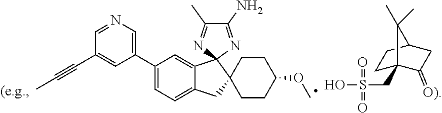

In certain such embodiments, the subject has a BMI of less than about 30 kg/m2 or less than about 25 kg/m2 after administration of the therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceuticallky acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

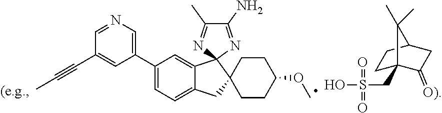

In certain embodiments, the method further comprises maintaining the reduced BMI, for at least one month, at least two months, at least three months, at least four months, at least five months, or at least six months. In certain embodiments, the method further comprises maintaining the reduced BMI, for at least one year, such as at least two years. The present application further provides a method of treating a condition or disorder associated with being overweight or obese comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

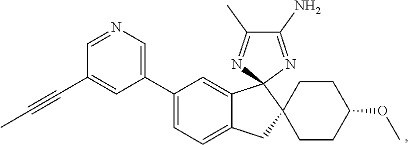 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

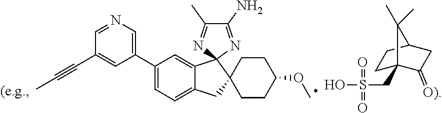

In certain such embodiments, the condition or disorder associated with being overweight or obese comprises a cardiac disorder (e.g., hypertension, dyslipidemia, ischemic heart disease, cardiomyopathy, cardiac infarction, stroke, venous thromboembolic disease and pulmonary hypertension), an endocrine disorder (e.g., type 2 diabetes and latent autoimmune diabetes in adults), a respiratory disorder (e.g., obesity-hypoventilation syndrome, asthma, and obstructive sleep apnea), a hepatic disorder (e.g., nonalcoholic fatty liver disease), a skeletal disorder (e.g., back pain and osteoarthritis of weight-bearing joints), a psychiatric disorder (e.g., weight-associated depression and anxiety), a metabolic disorder (e.g., Prader-Willi Syndrome and polycystic ovary syndrome), a sleeping disorder (e.g., sleep apnea), a reproductive disorder (e.g., sexual dysfunction, erectile dysfunction, infertility, obstetric complications, and fetal abnormalities), or a combination thereof. In certain embodiments, the method of treating a condition or disorder associated with being overweight or obese comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

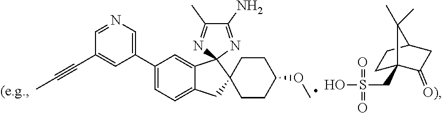

further comprises conjoint administration with an agent selected from sulfonylureas; meglitinides; biguanides; thiazolidinediones; alpha-glucosidase inhibitors; dipeptidylpeptidase-4 inhibitors; sodium:glucose co-transporter inhibitors; 11 beta hydroxycorticosterone dehydrogenase 1 inhibitors; glucagon-like peptide-1 analogs or mimetics; loop diuretics; potassium-sparing agents; peripheral agents; central alpha-agonists; alpha-blockers; beta-blockers; combined alpha- and beta-blockers; direct vasodilators; calcium antagonists; dihydropyridines; ACE inhibitors; Angiotensin II receptor blockers; renin inhibitors 1; soluble epoxide hydrolase inhibitors; statins; nitrates; inotropic agents; diuretics; anti-arrhythmic agents; thrombolytic agents; anti-platelet agents; anticoagulant agents; potassium; vasodilators; bronchodilators; anti-inflammatory agents; leukotriene blockers; anti-Ige agents; Modafinil; antioxidants; insulin sensitizers; hepatoprotectants, lipid-lowering agents; non-steroidal anti-inflammatory agents; COX-2 inhibitors; steroids; supplements; phosphodiesterase inhibitors; prostaglandin E analogs; alkaloids; Bromocriptine, Gonadotropin-releasing Hormone (GnRH); GnRH agonist; GnRH antagonist; Tamoxifen/nolvadex; gonadotropins; Human Chorionic Gonadotropin (HCG); Human Menopausal Gonadotropin (HmG); progesterone; recombinant follicle stimulating hormone (FSH); Urofollitropin; Follitropin alfa; Follitropin beta; human growth hormone (HGH); somatropin; weight loss agents (e.g., serotonin and noradrenergic re-uptake inhibitors, noradrenergic re-uptake inhibitors, selective serotonin re-uptake inhibitors, intestinal lipase inhibitors, orlistat, sibutramine, methamphetamine, ionamin, phentermine, bupropion, diethylpropion, phendimetrazine, benzphetermine, pramlintide, exenatide, liraglutide, and Topamax); combinations of synthetic estrogen and progesterone; Spironolactone; Eflornithine; Clomiphene; Bupivacaine hydrochloride; Dinoprostone PGE2; Meperidine HCl; Ferro-folic-500/iberet-folic-500; Meperidine; Methylergonovine maleate; Ropivacaine HCl; Nalbuphine HCl; Oxymorphone HCl; Oxytocin; Dinoprostone; Ritodrine; Scopolamine hydrobromide; Sufentanil citrate; Oxytocic; serotonin reuptake inhibitors; tricyclic antidepressants; monoamine oxidase inhibitors; psychostimulants; antipsychotics; mood stabilizers; benzodiazepines; and combinations thereof.

In certain embodiments of the methods of the application, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

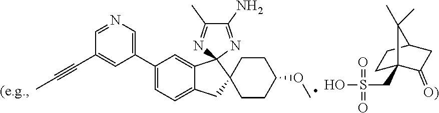

is administered orally once daily for at least 5 weeks, at least 10 weeks, at least 15 weeks, at least 20 weeks, at least 25 weeks, at least 30 weeks, at least 35 weeks, at least 40 weeks, at least 45 weeks, at least 50 weeks, at least 52 weeks, at least 75 weeks, at least 78 weeks, at least 100 weeks, or at least 104 weeks. In certain such embodiments, the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

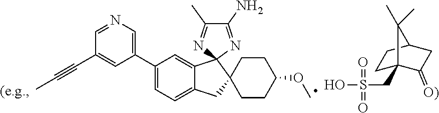

is administered at 20 mg or 50 mg daily.

Obesity and being overweight refer to an excess of fat in proportion to lean body mass. Excess fat accumulation is associated with increase in size (hypertrophy) as well as number (hyperplasia) of adipose tissue cells. Obesity is variously measured in terms of absolute weight, weight:height ratio, distribution of subcutaneous fat, and societal and esthetic norms. Generally, mammals having a weight that exceeds the 85th percentile are overweight, and those exceeding the 95th percentile are obese. A common measure of body fat is Body Mass Index (BMI). The BMI refers to the ratio of body weight (expressed in kilograms) to the square of height (expressed in meters). Body mass index may be accurately calculated using either of the formulas below:

Formula:weight(kg)/[height(m)]² or

Formula:703×weight(lbs)/[height(in)]².

In accordance with the U.S. Centers for Disease Control and Prevention (CDC), an “overweight” or “pre-obese” adult has a BMI of 25 kg/m2 to 29.9 kg/m2, and an “obese” adult has a BMI of 30 kg/m2 or greater. A BMI of 40 kg/m2 or greater is indicative of morbid obesity or extreme obesity. A BMI of 35 kg/m2 and one or more obesity-related conditions or co-morbidities is also indicative of a subject in need of treatment. For children, the definitions of overweight and obese take into account age and gender effects on body fat.

Increased waist measurement is another risk indicator for type 2 diabetes, the greater the waist measurement the greater the increased risk, and a substantial number of diabetic or pre-diabetic patients may have increased waist measurements (which may be but are not necessarily associated with a BMI of greater than 25). Thus, in some embodiments, patients contemplated herein have a waist measurement of more than 94 or 102 cm (adult men) or more than 80 or 88 cm (adult women).

BMI does not account for the fact that excess adipose can occur selectively in different parts of the body, and development of adipose tissue can be more dangerous to health in some parts of the body rather than in other parts of the body. For example, “central obesity”, typically associated with an “apple-shaped” body, results from excess adiposity especially in the abdominal region, including belly fat and visceral fat, and carries higher risk of co-morbidity than “peripheral obesity”, which is typically associated with a “pear-shaped” body resulting from excess adiposity especially on the hips. Measurement of waist/hip circumference ratio (WHR) can be used as an indicator of central obesity. A minimum WHR indicative of central obesity has been variously set, and a centrally obese adult typically has a WHR of about 0.85 or greater if female and about 0.9 or greater if male.

Compounds of the present application containing one or multiple asymmetrically substituted atoms may be isolated in optically active or racemic forms. It is well known in the art how to prepare optically active forms, such as by resolution of racemic forms, by synthesis from optically active starting materials, or by synthesis using optically active reagents.

In certain embodiments, compounds of the application may be racemic. For example, in embodiments of the application wherein a specific enantiomer of a compound is recited (e.g., (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

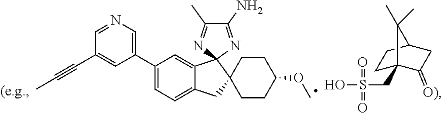

the application further contemplates the compound in its racemic form. In certain embodiments, compounds of the application may be enriched in one enantiomer. For example, a compound of the application may have greater than 30% ee, 40% ee, 50% ee, 60% ee, 70% ee, 80% ee, 90% ee, or even 95% or greater ee.

In certain embodiments, the therapeutic preparation may be enriched to provide predominantly one enantiomer of a compound (e.g., of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

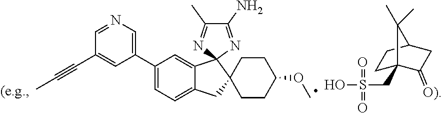

An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, compounds of the application may have more than one stereocenter. In certain such embodiments, compounds of the application may be enriched in one or more diastereomer. For example, a compound of the application may have greater than 30% de, 40% de, 50% de, 60% de, 70% de, 80% de, 90% de, or even 95% or greater de.

In certain embodiments, the therapeutic preparation may be enriched to provide predominantly one diastereomer of a compound (e.g., of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

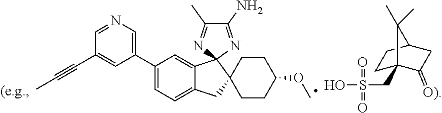

A diastereomerically enriched mixture may comprise, for example, at least 60 mol percent of one diastereomer, or more preferably at least 75, 90, 95, or even 99 mol percent.

A variety of compounds in the present application may exist in particular geometric or stereoisomeric forms. The present application takes into account all such compounds, including tautomers, cis- and trans-isomers, R- and S-enantiomers, diastereomers, (D)-isomers, (L)-isomers, the racemic mixtures thereof, and other mixtures thereof, as being covered within the scope of this application. All tautomeric forms are encompassed in the present application. Additional asymmetric carbon atoms may be present in a substituent such as an alkyl group. All such isomers, as well as mixtures thereof, are intended to be included in this application, unless the stereochemistry or isomeric form is specifically indicated.

The present application further includes all pharmaceutically acceptable isotopically labelled compounds (e.g., of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

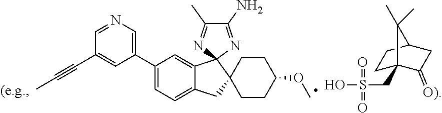

An “isotopically” or “radio-labelled” compound is a compound where one or more atoms are replaced or substituted by an atom having an atomic mass or mass number different from the atomic mass or mass number typically found in nature (i.e., naturally occurring). For example, in certain embodiments, in compounds (e.g., of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

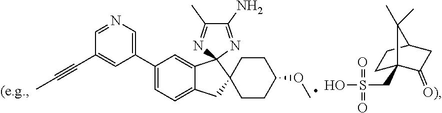

hydrogen atoms are replaced or substituted by one or more deuterium or tritium (e.g., hydrogen atoms on a C₁₋₆ alkyl or a C₁₋₆ alkoxy are replaced with deuterium, such as d₃-methoxy or 1,1,2,2-d₄-3-methylbutyl).

Certain isotopically labelled compounds (e.g., of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

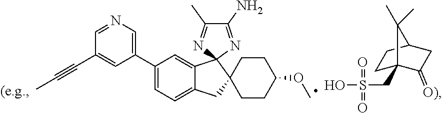

in the application, for example, those incorporating a radioactive isotope, are useful in drug and/or substrate tissue distribution studies. The radioactive isotopes tritium, i.e. ³H, and carbon 14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy.

Isotopically labelled compounds (e.g., of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

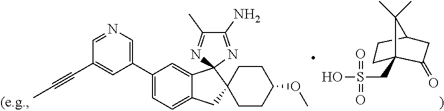 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

can generally be prepared by conventional techniques known to those skilled in the art or by processes analogous to those described in the accompanying examples using an appropriate isotopically labelled reagent in place of the non-labelled reagent previously employed. Suitable isotopes that may be incorporated in compounds of the present application include but are not limited to ²H (also written as D for deuterium), ³H (also written as T for tritium), ¹¹C, ¹³C,¹⁴C, ¹³N, ¹⁵N, ¹⁵O, ¹⁷O, ¹⁸O, ¹⁸F, ³⁵S, ³⁶Cl, ⁸²B r, ⁷⁵Br, ⁷⁶B r, ⁷⁷Br, ¹²³I, ¹²⁴I, ¹²⁵I, and ¹³¹I.

In certain embodiments, the present application provides a pharmaceutical preparation suitable for use in a human patient, comprising (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

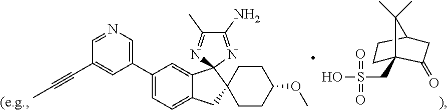 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

### Uses of the Compounds

Compounds of the present application may be administered orally, parenteral, buccal, vaginal, rectal, inhalation, insufflation, sublingually, intramuscularly, subcutaneously, topically, intranasally, intraperitoneally, intrathoracically, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient. The quantity of the compound to be administered will vary for the patient being treated and will vary from about 100 ng/kg of body weight to 100 mg/kg of body weight per day. For instance, dosages can be readily ascertained by those skilled in the art from this disclosure and the knowledge in the art. This, the skilled artisan can readily determine the amount of compound and optional additives, vehicles, and/or carrier in compositions and to be administered in methods of the application. In certain embodiments, the application relates to (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

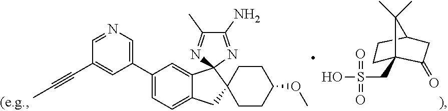 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

for use as a medicament, e.g., for treatment of any of the disorders disclosed herein.

In certain embodiments, the application relates to the use of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

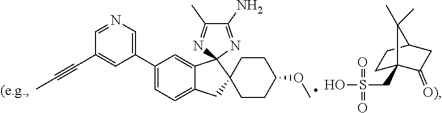

in the manufacture of a medicament for affecting weight loss, preventing weight gain, suppressing appetite, treating obesity, reducing BMI, or treating a condition or disorder associated with being overweight or obese in a subject. In certain such embodiments, the condition or disorder associated with being overweight or obese comprises a cardiac disorder (e.g., hypertension, dyslipidemia, ischemic heart disease, cardiomyopathy, cardiac infarction, stroke, venous thromboembolic disease and pulmonary hypertension), an endocrine disorder (e.g., type 2 diabetes and latent autoimmune diabetes in adults), a respiratory disorder (e.g., obesity-hypoventilation syndrome, asthma, and obstructive sleep apnea), a hepatic disorder (e.g., nonalcoholic fatty liver disease), a skeletal disorder (e.g., back pain and osteoarthritis of weight-bearing joints), a psychiatric disorder (e.g., weight-associated depression and anxiety), a metabolic disorder (e.g., Prader-Willi Syndrome and polycystic ovary syndrome), a sleeping disorder (e.g., sleep apnea), a reproductive disorder (e.g., sexual dysfunction, erectile dysfunction, infertility, obstetric complications, and fetal abnormalities), or a combination thereof.

In certain embodiments, the application relates to a method of treating or preventing a condition or disorder associated with being overweight or obese in a subject in a mammal, such as a human being, comprising administering to said patient a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

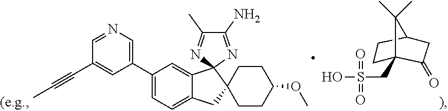 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

and at least one agent selected from sulfonylureas; meglitinides; biguanides; thiazolidinediones; alpha-glucosidase inhibitors; dipeptidylpeptidase-4 inhibitors; sodium:glucose co-transporter inhibitors; 11 beta hydroxycorticosterone dehydrogenase 1 inhibitors; glucagon-like peptide-1 analogs or mimetics; loop diuretics; potassium-sparing agents; peripheral agents; central alpha-agonists; alpha-blockers; beta-blockers; combined alpha- and beta-blockers; direct vasodilators; calcium antagonists; dihydropyridines; ACE inhibitors; Angiotensin II receptor blockers; renin inhibitors 1; soluble epoxide hydrolase inhibitors; statins; nitrates; inotropic agents; diuretics; anti-arrhythmic agents; thrombolytic agents; anti-platelet agents; anticoagulant agents; potassium; vasodilators; bronchodilators; anti-inflammatory agents; leukotriene blockers; anti-Ige agents; Modafinil; antioxidants; insulin sensitizers; hepatoprotectants, lipid-lowering agents; non-steroidal anti-inflammatory agents; COX-2 inhibitors; steroids; supplements; phosphodiesterase inhibitors; prostaglandin E analogs; alkaloids; Bromocriptine, Gonadotropin-releasing Hormone (GnRH); GnRH agonist; GnRH antagonist; Tamoxifen/nolvadex; gonadotropins; Human Chorionic Gonadotropin (HCG); Human Menopausal Gonadotropin (HmG); progesterone; recombinant follicle stimulating hormone (FSH); Urofollitropin; Follitropin alfa; Follitropin beta; human growth hormone (HGH); somatropin; weight loss agents (e.g., serotonin and noradrenergic re-uptake inhibitors, noradrenergic re-uptake inhibitors, selective serotonin re-uptake inhibitors, intestinal lipase inhibitors, orlistat, sibutramine, methamphetamine, ionamin, phentermine, bupropion, diethylpropion, phendimetrazine, benzphetermine, pramlintide, exenatide, liraglutide, and Topamax); combinations of synthetic estrogen and progesterone; Spironolactone; Eflornithine; Clomiphene; Bupivacaine hydrochloride; Dinoprostone PGE2; Meperidine HCl; Ferro-folic-500/iberet-folic-500; Meperidine; Methylergonovine maleate; Ropivacaine HCl; Nalbuphine HCl; Oxymorphone HCl; Oxytocin; Dinoprostone; Ritodrine; Scopolamine hydrobromide; Sufentanil citrate; Oxytocic; serotonin reuptake inhibitors; tricyclic antidepressants; monoamine oxidase inhibitors; psychostimulants; antipsychotics; mood stabilizers; benzodiazepines; and combinations thereof. In certain embodiments, the application relates to a method of treating or preventing a condition or disorder associated with being overweight or obese in a subject in a mammal, such as a human being, comprising administering to said patient a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

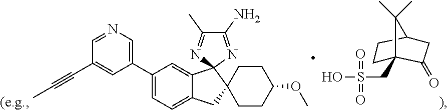 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

and at least one weight loss agent selected from the group consisting of serotonin and noradrenergic re-uptake inhibitors; noradrenergic re-uptake inhibitors; selective serotonin re-uptake inhibitors; and intestinal lipase inhibitors (e.g., orlistat, sibutramine, methamphetamine, ionamin, phentermine, bupropion, diethylpropion, phendimetrazine, benzphetermine, and topamax).

In certain embodiments, the application relates to a pharmaceutical composition comprising (1) (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

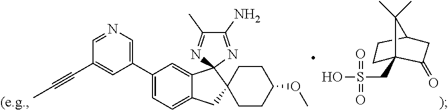 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

(2) at least one agent selected from the group consisting of agent selected from sulfonylureas; meglitinides; biguanides; thiazolidinediones; alpha-glucosidase inhibitors; dipeptidylpeptidase-4 inhibitors; sodium:glucose co-transporter inhibitors; 11 beta hydroxycorticosterone dehydrogenase 1 inhibitors; glucagon-like peptide-1 analogs or mimetics; loop diuretics; potassium-sparing agents; peripheral agents; central alpha-agonists; alpha-blockers; beta-blockers; combined alpha- and beta-blockers; direct vasodilators; calcium antagonists; dihydropyridines; ACE inhibitors; Angiotensin II receptor blockers; renin inhibitors 1; soluble epoxide hydrolase inhibitors; statins; nitrates; inotropic agents; diuretics; anti-arrhythmic agents; thrombolytic agents; anti-platelet agents; anticoagulant agents; potassium; vasodilators; bronchodilators; anti-inflammatory agents; leukotriene blockers; anti-Ige agents; Modafinil; antioxidants; insulin sensitizers; hepatoprotectants, lipid-lowering agents; non-steroidal anti-inflammatory agents; COX-2 inhibitors; steroids; supplements; phosphodiesterase inhibitors; prostaglandin E analogs; alkaloids; Bromocriptine, Gonadotropin-releasing Hormone (GnRH); GnRH agonist; GnRH antagonist; Tamoxifen/nolvadex; gonadotropins; Human Chorionic Gonadotropin (HCG); Human Menopausal Gonadotropin (HmG); progesterone; recombinant follicle stimulating hormone (FSH); Urofollitropin; Follitropin alfa; Follitropin beta; human growth hormone (HGH); somatropin; weight loss agents (e.g., serotonin and noradrenergic re-uptake inhibitors, noradrenergic re-uptake inhibitors, selective serotonin re-uptake inhibitors, intestinal lipase inhibitors, orlistat, sibutramine, methamphetamine, ionamin, phentermine, bupropion, diethylpropion, phendimetrazine, benzphetermine, pramlintide, exenatide, liraglutide, and Topamax); combinations of synthetic estrogen and progesterone; Spironolactone; Eflornithine; Clomiphene; Bupivacaine hydrochloride; Dinoprostone PGE2; Meperidine HCl; Ferro-folic-500/iberet-folic-500; Meperidine; Methylergonovine maleate; Ropivacaine HCl; Nalbuphine HCl; Oxymorphone HCl; Oxytocin; Dinoprostone; Ritodrine; Scopolamine hydrobromide; Sufentanil citrate; Oxytocic; serotonin reuptake inhibitors; tricyclic antidepressants; monoamine oxidase inhibitors; psychostimulants; antipsychotics; mood stabilizers; benzodiazepines; and combinations thereof, and (3) pharmaceutically acceptable excipients, carriers or diluents. In certain embodiments, the application relates to a pharmaceutical composition comprising (1) (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro [cyclohexane-1,2′-indene-1′,2″-imidazol]-4″-amine

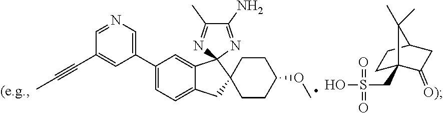

(2) at least one weight loss agent, such as a weight loss agent selected from the group consisting of serotonin and noradrenergic re-uptake inhibitors; noradrenergic re-uptake inhibitors; selective serotonin re-uptake inhibitors; and intestinal lipase inhibitors (e.g., orlistat, sibutramine, methamphetamine, ionamin, phentermine, bupropion, diethylpropion, phendimetrazine, benzphetermine, and topamax), and (3) pharmaceutically acceptable excipients, carriers or diluents.

In the treatment of any of the disorders disclosed herein, (1r,1′R,4R)-4-methoxy-5″-methyl-6′-[5-(prop-1-yn-1-yl)pyridin-3-yl]-3′H-dispiro[cyclohexane-1,2′-indene-1′,2′-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

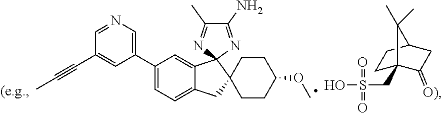

may be conjointly administered with other conventional therapeutic agents in one or more of the methods or in treating one or more disease conditions referred to herein.

In certain embodiments, (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

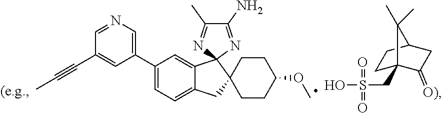

may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either simultaneously, sequentially, or by separate dosing of the individual components of the treatment. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

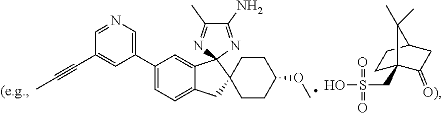

with one or more additional therapeutic agent(s) provides improved efficacy relative to each individual administration of the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

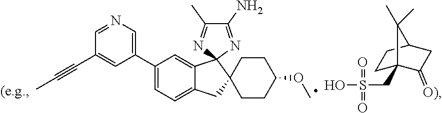

or the one or more additional therapeutic agent(s). In certain such embodiments, the conjoint administration provides an additive effect, wherein an additive effect refers to the sum of each of the effects of individual administration of the compound of the application and the one or more additional therapeutic agent(s).

Type 2 diabetes has been associated with obesity. Certain complications of type 2 diabetes, e.g., disability and premature death, can be prevented, ameliorated, or eliminated by sustained weight loss (Astrup, A. Pub Health Nutr (2001) 4:499-515). Accordingly, the present application provides methods of treating or reducing the risk of type 2 diabetes, reducing the complications of type 2 diabetes, and/or treating or preventing complications associated with type 2 diabetes comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2′-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

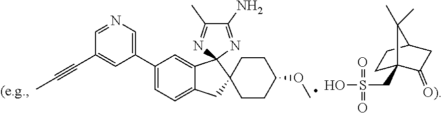

In certain such embodiments, the complications of type 2 diabetes comprise disability or premature death. Agents administered to treat type 2 diabetes in regimens and at dosages known in the art include sulfonylureas (e.g., Chlorpropamide, Glipizide, Glyburide, Glimepiride); meglitinides (e.g., Repaglinide and Nateglinide); biguanides (e.g., Metformin); thiazolidinediones (Rosiglitazone, Troglitazone, and Pioglitazone); and alpha-glucosidase inhibitors (e.g., Acarbose and Meglitol). Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amino

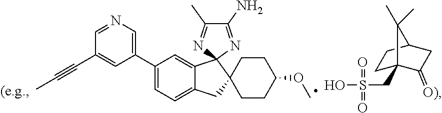

in combination with at least one of these agents results in weight loss, and provides an increased benefit in ameliorating, arresting development of or eliminating type 2 diabetes in a subject compared to administration of one of these agents alone.

Cardiac disorders and conditions, for example hypertension, dyslipidemia, ischemic heart disease, cardiomyopathy, cardiac infarction, stroke, venous thromboembolic disease and pulmonary hypertension, have been linked to being overweight or obese. For example, hypertension has been linked to obesity because excess adipose tissue secretes substances that are acted on by the kidneys, resulting in hypertension. Additionally, with obesity there are generally higher amounts of insulin produced (because of the excess adipose tissue) and this excess insulin also elevates blood pressure. A major treatment option of hypertension is weight loss. Accordingly, the present application provides methods of treating or reducing the risk of cardiac disorders and conditions (e.g., hypertension, dyslipidemia, ischemic heart disease, cardiomyopathy, cardiac infarction, stroke, venous thromboembolic disease and pulmonary hypertension) comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

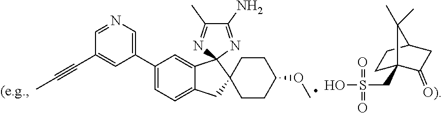

Agents administered to treat hypertension in regimens and at dosages known in the art include Chlorthalidone; Hydrochlorothiazide; Indapamide, Metolazone; loop diuretics (e.g., Bumetanide, Ethacrynic acid, Furosemide, Lasix, Torsemide); potassium-sparing agents (e.g., Amiloride hydrochloride, Spironolactone, and Triamterene); peripheral agents (e.g., Reserpine); central alpha-agonists (e.g., Clonidine hydrochloride, Guanabenz acetate, Guanfacine hydrochloride, and Methyldopa); alpha-blockers (e.g., Doxazosin mesylate, Prazosin hydrochloride, and Terazosin hydrochloride); beta-blockers (e.g., Acebutolol, Atenolol, Betaxolol, Bisoprolol fumarate, Carteolol hydrochloride, Metoprolol tartrate, Metoprolol succinate, Nadolol, Penbutolol sulfate, Pindolol, Propranolol hydrochloride, and Timolol maleate); combined alpha- and beta-blockers (e.g., Carvedilol and Labetalol hydrochloride); direct vasodilators (e.g., Hydralazine hydrochloride and Minoxidil); calcium antagonists (e.g., Diltiazem hydrochloride and Verapamil hydrochloride); dihydropyridines (e.g., Amlodipine besylate, Felodipine, Isradipine, Nicardipine, Nifedipine, and Nisoldipine); ACE inhibitors (benazepril hydrochloride, Captopril, Enalapril maleate, Fosinopril sodium, Lisinopril, Moexipril, Quinapril hydrochloride, Ramipril, Trandolapril); Angiotensin II receptor blockers (e.g., Losartan potassium, Valsartan, and Irbesartan); and combinations thereof. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

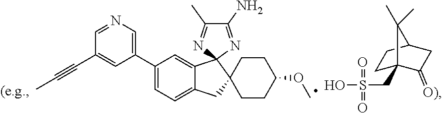

in combination with at least one of these agents results in weight loss, and provides an increased benefit in ameliorating, arresting development of or eliminating hypertension in a subject compared to administration of one of these agents alone.

Carr et al. (The Journal of Clinical Endocrinology & Metabolism (2004) Vol. 89, No. 6 2601-2607) discusses a link between being overweight or obese and dyslipidemia. Accordingly, the present application provides methods of treating or reducing the risk of dyslipidemia comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

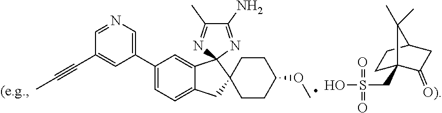

Dyslipidemia is typically treated with statins in regimens and at dosages known in the art. Statins, HMG-CoA reductase inhibitors, slow down production of cholesterol in a subject and/or remove cholesterol buildup from arteries. Statins include mevastatin, lovastatin, pravastatin, simvastatin, velostatin, dihydrocompactin, fluvastatin, atorvastatin, dalvastatin, carvastatin, crilvastatin, bevastatin, cefvastatin, rosuvastatin, pitavastatin, and glenvastatin. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

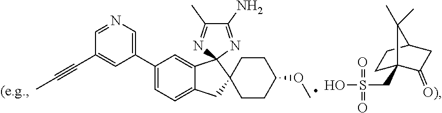

in combination with at least one of these agents results in weight loss, and provides an increased benefit in ameliorating, arresting development of or eliminating dyslipidemia in a subject compared to administration of one of these agents alone.

Eckel (Circulation (1997) 96:3248-3250) discusses a link between being overweight or obese and ischemic heart disease. Accordingly, the present application provides methods of treating or reducing the risk of ischemic heart disease comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

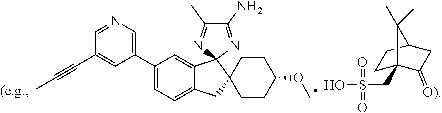

Agents administered to treat ischemic heart disease in regimens and at dosages known in the art include statins, nitrates (e.g., Isosorbide Dinitrate and Isosorbide Mononitrate), beta-blockers, and calcium channel antagonists. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

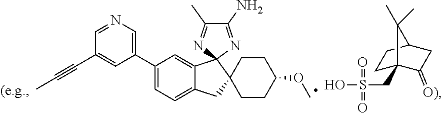

in combination with at least one of these agents results in weight loss, and provides an increased benefit in ameliorating, arresting development of or eliminating ischemic heart disease in a subject compared to administration of one of these agents alone.

Wong et al. (Nature Clinical Practice Cardiovascular Medicine (2007) 4:436-443) discusses a link between being overweight or obese and cardiomyopathy. Accordingly, the present application provides methods of treating or reducing the risk of cardiomyopathy comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

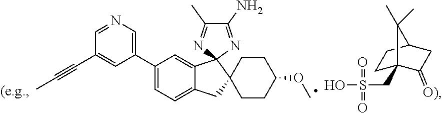

Agents administered to treat cardiomyopathy in regimens and at dosages known in the art include inotropic agents (e.g., Digoxin), diuretics (e.g., Furosemide), ACE inhibitors, calcium antagonists, anti-arrhythmic agents (e.g., Sotolol, Amiodarone and Disopyramide), and beta-blockers. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

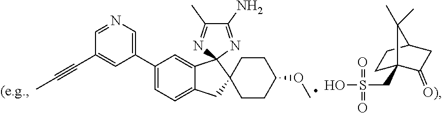

in combination with at least one of these agents results in weight loss, and provides an increased benefit in ameliorating, arresting development of or eliminating cardiomyopathy in a subject compared to administration of one of these agents alone.

Yusef et al. (Lancet (2005) 366(9497):1640-1649) discusses a link between being overweight or obese and cardiac infarction. Accordingly, the present application provides methods of treating or reducing the risk of cardiac infarction comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

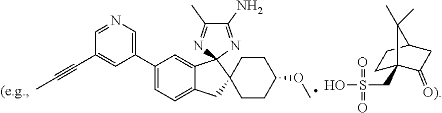

Agents administered to treat cardiac infarction in regimens and at dosages known in the art include ACE inhibitors, Angiotensin II receptor blockers, direct vasodilators, beta blockers, anti-arrhythmic agents and thrombolytic agents (e.g., Alteplase, Retaplase, Tenecteplase, Anistreplase, and Urokinase). Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

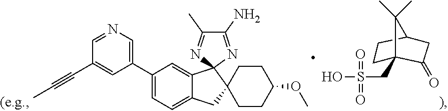 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating cardiac infarction in a subject compared to administration of one of these agents alone.

Suk et al. (Stroke (2003) 34:1586-1592) discusses a link between being overweight or obese and strokes. Accordingly, the present application provides methods of treating or reducing the risk of stroke comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

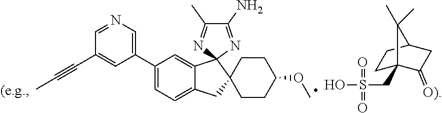

Agents administered to treat strokes in regimens and at dosages known in the art include anti-platelet agents (e.g., Aspirin, Clopidogrel, Dipyridamole, and Ticlopidine), anticoagulant agents (e.g., Heparin), and thrombolytic agents. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

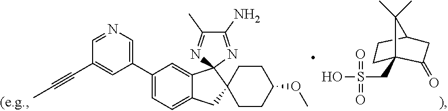 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating strokes in a subject compared to administration of one of these agents alone.

Stein et al. (The American Journal of Medicine (2005) 18(9):978-980) discusses a link between being overweight or obese and venous thromboembolic disease. Accordingly, the present application provides methods of treating or reducing the risk of venous thromboembolic disease comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

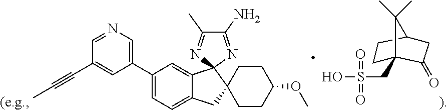 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

Agents administered to treat venous thromboembolic disease in regimens and at dosages known in the art include anti-platelet agents, anticoagulant agents, and thrombolytic agents. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

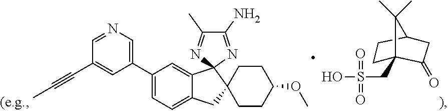 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating venous thromboembolic disease in a subject compared to administration of one of these agents alone.

Sztrymf et al. (Rev Pneumol Clin (2002) 58(2):104-10) discusses a link between being overweight or obese and pulmonary hypertension. Accordingly, the present application provides methods of treating or reducing the risk of pulmonary hypertension comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

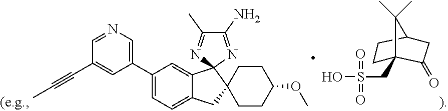 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

Agents administered to treat pulmonary hypertension in regimens and at dosages known in the art include inotropic agents, anticoagulant agents, diuretics, potassium (e.g., K-dur), vasodilators (e.g., Nifedipine and Diltiazem), Bosentan, Epoprostenol, and Sildenafil. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

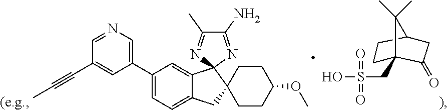 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating pulmonary hypertension in a subject compared to administration of one of these agents alone.

Respiratory disorders and conditions such as obesity-hypoventilation syndrome, asthma, and obstructive sleep apnea, have been linked to being overweight or obese. Elamin (Chest (2004) 125:1972-1974) discusses a link between being overweight or obese and asthma. Accordingly, the present application provides methods of treating or reducing the risk of respiratory disorders and conditions (e.g., obesity-hypoventilation syndrome, asthma, and obstructive sleep apnea) comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

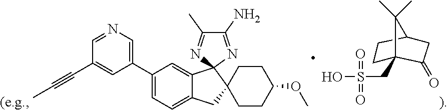 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

Agents administered to treat asthma in regimens and at dosages known in the art include bronchodilators, anti-inflammatory agents, leukotriene blockers, and anti-Ige agents. Particular asthma agents include Zafirlukast, Flunisolide, Triamcinolone, Beclomethasone, Terbutaline, Fluticasone, Formoterol, Beclomethasone, Salmeterol, Theophylline, and Xopenex. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

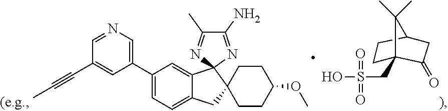 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating asthma in a subject compared to administration of one of these agents alone. Kessler et al. (Eur Respir J (1996) 9:787-794) discusses a link between being overweight or obese and obstructive sleep apnea. Agents administered to treat sleep apnea in regimens and at dosages known in the art include Modafinil and amphetamines. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

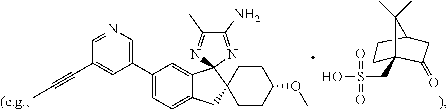 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating obstructive sleep apnea in a subject compared to administration of one of these agents alone.

Hepatic disorders and conditions, such as nonalcoholic fatty liver disease, have been linked to being overweight or obese. Tolman et al. (Ther Clin Risk Manag (2007) 6:1153-1163) discusses a link between being overweight or obese and nonalcoholic fatty liver disease. Accordingly, the present application provides methods of treating or reducing the risk of hepatic disorders and conditions (e.g., nonalcoholic fatty liver disease) comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

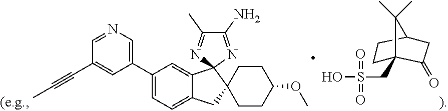 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

Agents administered to treat nonalcoholic fatty liver disease in regimens and at dosages known in the art include antioxidants (e.g., Vitamins E and C), insulin sensitizers (Metformin, Pioglitazone, Rosiglitazone, and Betaine), hepatoprotectants, and lipid-lowering agents. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

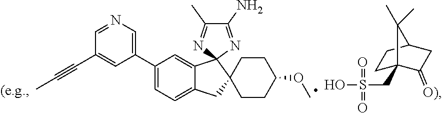

in combination with at least one of these agents results in weight loss, and provides an increased benefit in ameliorating, arresting development of or eliminating nonalcoholic fatty liver disease in a subject compared to administration of one of these agents alone.

Skeletal disorders and conditions, such as back pain and osteoarthritis of weight-bearing joints, have been linked to being overweight or obese. van Saase (J Rheumatol (1988) 15(7):1152-1158) discusses a link between being overweight or obese and osteoarthritis of weight-bearing joints. Accordingly, the present application provides methods of treating or reducing the risk of skeletal disorders and conditions (e.g., back pain and osteoarthritis of weight-bearing joints) comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

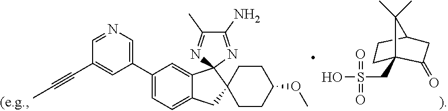 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

Agents administered to treat osteoarthritis of weight-bearing joints in regimens and at dosages known in the art include Acetaminophen, non-steroidal anti-inflammatory agents (e.g., Ibuprofen, Etodolac, Oxaprozin, Naproxen, Diclofenac, and Nabumetone), COX-2 inhibitors (e.g., Celecoxib), steroids, supplements (e.g. glucosamine and chondroitin sulfate), and artificial joint fluid. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

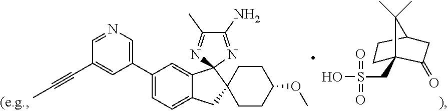 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating osteoarthritis of weight-bearing joints in a subject compared to administration of one of these agents alone.

Metabolic disorders and conditions, for example, Prader-Willi Syndrome and polycystic ovary syndrome, have been linked to being overweight or obese. Cassidy (Journal of Medical Genetics (1997) 34:917-923) discusses a link between being overweight or obese and Prader-Willi Syndrome. Accordingly, the present application provides methods of treating or reducing the risk of metabolic disorders and conditions (e.g., Prader-Willi Syndrome and polycystic ovary syndrome) comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

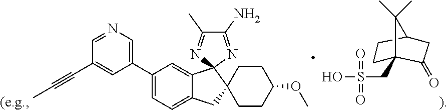 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

Agents administered to treat Prader-Willi Syndrome in regimens and at dosages known in the art include human growth hormone (HGH), somatropin, and weight loss agents (e.g., Orlistat, Sibutramine, Methamphetamine, Ionamin, Phentermine, Bupropion, Diethylpropion, Phendimetrazine, Benzphetermine, and Topamax). Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

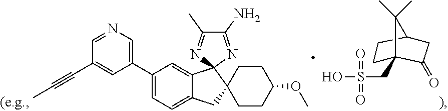 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating Prader-Willi Syndrome in a subject compared to administration of one of these agents alone. Hoeger (Obstetrics and Gynecology Clinics of North America (2001) 28(1):85-97) discusses a link between being overweight or obese and polycystic ovary syndrome. Agents administered to treat polycystic ovary syndrome in regimens and at dosages known in the art include insulin-sensitizers, combinations of synthetic estrogen and progesterone, Spironolactone, Eflornithine, and Clomiphene. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

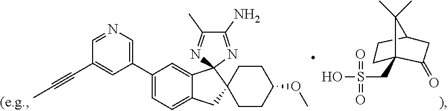 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating polycystic ovary syndrome in a subject compared to administration of one of these agents alone.

Reproductive disorders and conditions such as sexual dysfunction, erectile dysfunction, infertility, obstetric complications, and fetal abnormalities, have been linked to being overweight or obese. Larsen et al. (Int J Obes (Lond) (2007) 8:1189-1198) discusses a link between being overweight or obese and sexual dysfunction. Chung et al. (Eur Urol (1999) 36(1):68-70) discusses a link between being overweight or obese and erectile dysfunction. Accordingly, the present application provides methods of treating or reducing the risk of reproductive disorders and conditions (e.g., sexual dysfunction, erectile dysfunction, infertility, obstetric complications, and fetal abnormalities) comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

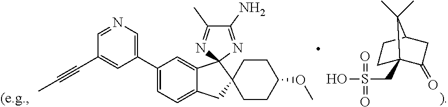 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

Agents administered to treat erectile dysfunction in regimens and at dosages known in the art include phosphodiesterase inhibitors (e.g., Tadalafil, Sildenafil citrate, and Vardenafil), prostaglandin E analogs (e.g., Alprostadil), alkaloids (e.g., Yohimbine), and testosterone. Administering (1r,1′R,4R)-4-methoxy-5″-methyl-6′-[5-(prop-1-yn-1-yl)pyridin-3-yl]-3′H-dispiro[cyclohexane-1,2′-indene-1′,2′-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

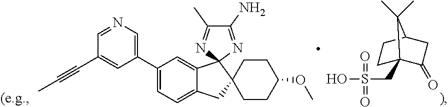 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating erectile dysfunction in a subject compared to administration of one of these agents alone. Pasquali et al. (Hum Reprod (1997) 1:82-87) discusses a link between being overweight or obese and infertility. Agents administered to treat infertility in regimens and at dosages known in the art include Clomiphene, Clomiphene citrate, Bromocriptine, Gonadotropin-releasing Hormone (GnRH), GnRH agonist, GnRH antagonist, Tamoxifen/nolvadex, gonadotropins, Human Chorionic Gonadotropin (HCG), Human Menopausal Gonadotropin (HmG), progesterone, recombinant follicle stimulating hormone (FSH), Urofollitropin, Heparin, Follitropin alfa, and Follitropin beta. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

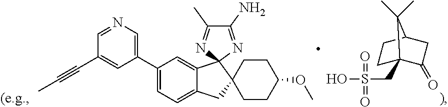 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating infertility in a subject compared to administration of one of these agents alone. Weiss et al. (American Journal of Obstetrics and Gynecology (2004) 190(4):1091-1097) discusses a link between being overweight or obese and obstetric complications. Agents administered to treat obstetric complications in regimens and at dosages known in the art include Bupivacaine hydrochloride, Dinoprostone PGE2, Meperidine HCl, Ferro-folic-500/iberet-folic-500, Meperidine, Methylergonovine maleate, Ropivacaine HCl, Nalbuphine HCl, Oxymorphone HCl, Oxytocin, Dinoprostone, Ritodrine, Scopolamine hydrobromide, Sufentanil citrate, and Oxytocic. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

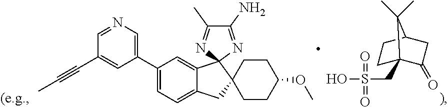 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating obstetric complications in a subject compared to administration of one of these agents alone.

Psychiatric disorders and conditions, for example, weight-associated depression and anxiety, have been linked to being overweight or obese. Dixson et al. (Arch Intern Med (2003) 163:2058-2065) discusses a link between being overweight or obese and depression. Accordingly, the present application provides methods of treating or reducing the risk of psychiatric disorders and conditions (e.g., weight-associated depression and anxiety), comprising administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

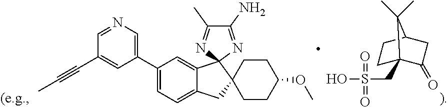 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

Agents administered to treat depression in regimens and at dosages known in the art include serotonin reuptake inhibitors (e.g., Fluoxetine, Escitalopram, Citalopram, Paroxetine, Sertraline, and Venlafaxine); tricyclic antidepressants (e.g., Amitriptyline, Amoxapine, Clomipramine, Desipramine, Dosulepin hydrochloride, Doxepin, Imipramine, Iprindole, Lofepramine, Nortriptyline, Opipramol, Protriptyline, and Trimipramine); monoamine oxidase inhibitors (e.g., Isocarboxazid, Moclobemide, Phenelzine, Tranylcypromine, Selegiline, Rasagiline, Nialamide, Iproniazid, Iproclozide, Toloxatone, Linezolid, Dienolide kavapyrone desmethoxyyangonin, and Dextroamphetamine); psychostimulants (e.g., Amphetamine, Methamphetamine, Methylphenidate, and Arecoline); antipsychotics (e.g., Butyrophenones, Phenothiazines, Thioxanthenes, Clozapine, Olanzapine, Risperidone, Quetiapine, Ziprasidone, Amisulpride, Paliperidone, Symbyax, Tetrabenazine, and Cannabidiol); and mood stabilizers (e.g., Lithium carbonate, Valproic acid, Divalproex sodium, Sodium valproate, Lamotrigine, Carbamazepine, Gabapentin, Oxcarbazepine, and Topiramate). Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

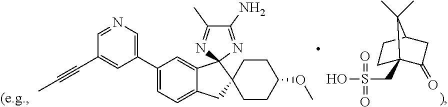 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating depression in a subject compared to administration of one of these agents alone. Simon et al. (Archives of General Psychiatry (2006) 63(7):824-830) discusses a link between being overweight or obese and anxiety. Agents administered to treat anxiety in regimens and at dosages known in the art include serotonin reuptake inhibitors, mood stabilizers, benzodiazepines (e.g., Alprazolam, Clonazepam, Diazepam, and Lorazepam), tricyclic antidepressants, monoamine oxidase inhibitors, and beta-blockers. Administering (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

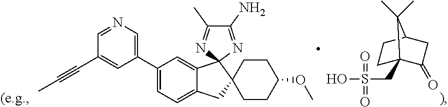 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in combination with at least one of these agents results in weight loss, and provides an increased benefit in ameliorating, arresting development of or eliminating anxiety in a subject compared to administration of one of these agents alone.

### Definitions

The definitions set forth in this application are intended to clarify terms used throughout this application.

The term “herein” means the entire application.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The present application includes prodrugs of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

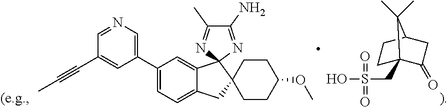 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

The term “prodrug” is intended to encompass compounds which, under physiologic conditions, are converted into the therapeutically active agents of the present application (e.g., (1r,1′R,4R)-4-methoxy-5″-methyl-6′-[5-(prop-1-yn-1-yl)pyridin-3-yl]-3′H-dispiro[cyclohexane-1,2′-indene-1′,2′-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

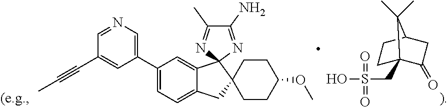 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

A common method for making a prodrug is to include one or more selected moieties which are hydrolyzed under physiologic conditions to yield the desired molecule. In certain embodiments, the prodrug is converted by an enzymatic activity of the host animal. For example, a prodrug with a nitro group on an aromatic ring could be reduced by reductase to generate the desired amino group of the corresponding active compound in vivo. In another example, functional groups such as a hydroxyl, carbonate, or carboxylic acid in the parent compound are presented as an ester, which could be cleaved by esterases. Additionally, amine groups in the parent compounds are presented in, but not limited to, carbamate, N-alkylated or N-acylated forms (Simplício et al, “Prodrugs for Amines,” Molecules, (2008), 13:519-547). In certain embodiments, (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

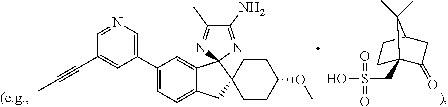 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

in a formulation represented above can be replaced with the corresponding suitable prodrug.

The present application includes metabolites of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

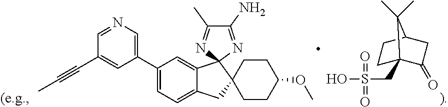 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

The term “metabolite” is intended to encompass compounds that are produced by metabolism/biochemical modification of the parent compound under physiological conditions, e.g. through certain enzymatic pathway. For example, an oxidative metabolite is formed by oxidation of the parent compound during metabolism, such as the oxidation of a pyridine ring to pyridine-N-oxide. In another example, an oxidative metabolite is formed by demethylation of a methoxy group to result in a hydroxyl group.

### Pharmaceutical Compositions

The compositions and methods of the present application may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the application and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the application. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the application. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the application, with the carrier and, optionally, one or more accessory ingredients. In general, the formulations are prepared by uniformly and intimately bringing into association a compound of the present application with liquid carriers, or finely divided solid carriers, or both, and then, if necessary, shaping the product.

Formulations of the application suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present application as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present application to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this application. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the application include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this application, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the application. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the application will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present application, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

This application includes the use of pharmaceutically acceptable salts of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine in the compositions and methods of the present application. The term “pharmaceutically acceptable salts” includes salts of the active compounds which are prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, such as an amine, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, camphorsulfonic and the like. In certain embodiments, the pharmaceutically acceptable salt is a hydrochloride salt. In certain embodiments, the pharmaceutically acceptable salt is a camsylate salt. In certain embodiments, contemplated salts of the compounds include, but are not limited to, alkyl, dialkyl, trialkyl or tetra-alkyl ammonium salts. In certain embodiments, contemplated salts of compounds include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of compounds include, but are not limited to, Li, Na, Ca, K, Mg, Zn or other metal salts. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present application may contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present application.

The compounds of the application, (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

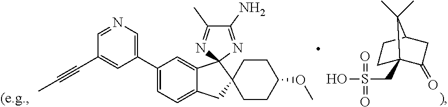 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

can also exist as various solvates, such as with water (also known as hydrates), methanol, ethanol, dimethylformamide, diethyl ether, acetamide, and the like. Mixtures of such solvates can also be prepared. The source of such solvate can be from the solvent of crystallization, inherent in the solvent of preparation or crystallization, or adventitious to such solvent.

The compounds of the application, (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

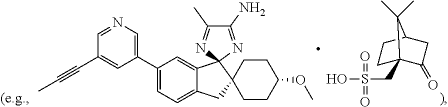 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

can also exist as various polymorphs, pseudo-polymorphs, or in amorphous state. As used herein, the term “polymorph” refers to different crystalline forms of the same compound and other solid state molecular forms including pseudo-polymorphs, such as hydrates, solvates, or salts of the same compound. Different crystalline polymorphs have different crystal structures due to a different packing of molecules in the lattice, as a result of changes in temperature, pressure, or variations in the crystallization process. Polymorphs differ from each other in their physical properties, such as x-ray diffraction characteristics, stability, melting points, solubility, or rates of dissolution in certain solvents. Thus crystalline polymorphic forms are important aspects in the development of suitable dosage forms in pharmaceutical industry.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In certain embodiments, the application comprises a method for conducting a pharmaceutical business, by determining an appropriate formulation and dosage of a compound of the application for treating or preventing any of the diseases or conditions as described herein, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the application relates to a method for conducting a pharmaceutical business by determining an appropriate formulation and dosage of a compound of the application for treating or preventing any of the disease or conditions as described herein, and licensing, to a third party, the rights for further development and sale of the formulation.

### Methods of Preparation

The compounds of the present application, i.e., (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, and pharmaceutically acceptable salts thereof, such as the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

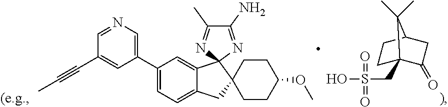 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

can be prepared as provided in WO 2012/087237, WO 2013/190302, PCT/EP2018/086439, and PCT/EP2018/086784.

### EXAMPLES

Below follows a number of non-limiting examples of compounds of the application.

### Example 1

A Phase 2/3, multicenter, randomized, 104-week, double-blind, placebo-controlled, global study of lanabecestat in patients with early Alzheimer's Disease (AD), defined as the continuum of patients with mild cognitive impairment (MCI) due to AD and patients diagnosed with mild AD dementia.

Lanabecestat, i.e., the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

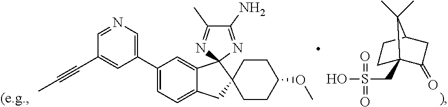 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

was provided orally once daily for 104 weeks at 20 mg or 50 mg and compared to the results for once daily administration of placebo.

The key inclusion criteria for the study were as follows:
Population aged 55 to 85 years old;
Mild AD: meet National Institute on Aging and the Alzheimer's Association (NIA-AA) criteria with a Clinical Dementia Rating Scale-Global Score (CDR-GS) of 0.5 or 1;
MCI: meet NIA-AA criteria with a CDR-GS of 0.5;
CDR memory box score ≥0.5;
Mini-Mental State Examination (MMSE)≥20;
Repeatable Battery for the Assessment of Neuropsychological Status Delayed Memory Index (RBANS DMI)≤85;
Study partner;
Amyloid positive
   Cerebrospinal fluid; or
   Florbetapir amyloid Positron Emission Tomography (PET); or
   Historical amyloid PET.

The key exclusion criteria for the study were as follows:
Unstable medical conditions or medications;
Magnetic Resonance Imaging (MRI): >5 microhemorrhages, significant cerebrovascular pathology, or other pathologies;
QT interval adjusted for heart rate using the Fridericia formula (QTcF) >470 ms;
History of vitiligo and/or current evidence of post-inflammatory hypo-pigmentation or exposure to depigmenting agents.

The baseline demographics for subjects enrolled in the study are summarized in Table 1.

TABLE 1
Baseline Demographics

| | | Lanabecestat | Lanabecestat |
|---|---|---|---|
| | Placebo | 20 mg | 50 mg |
| Demographic | (N = 740) | (N = 739) | (N = 739) |
| Sex, % Female | 53.8% | 53.5% | 52.0% |
| Age, mean | 71.4 | 71.2 | 71.2 |
| Race, % Asian | 11.5% | 11.5% | 13.8% |
| % Black or AA | 0.7% | 0.7% | 0.8% |
| % White | 80.8% | 82.4% | 80.2% |
| % Other^{a} | 7.0% | 5.4% | 5.2% |
| Ethnicity, % Hispanic/Latino | 6.4% | 3.8%* | 3.6%* |
| Education, % 13 years+ | 53.6% | 54.2% | 57.5% |
| BMI (kg/m²), mean | 25.4 | 25.2 | 25.2 |
| Employment, % paid | 9.0% | 9.8% | 10.5% |
| employment | | | |
| APOE 4, % carrier | 66.8% | 69.3% | 70.0% |
| Family History AD, % 1st | 41.2% | 42.2% | 44.4% |
| Degree, Yes | | | |
| AChEI use, % (n) | 66.9% | 66.2% | 68.7% |

Abbreviations: AA = African American; AChEI = acetylcholinesterase inhibitor; AD = Alzheimer's disease; APOE 4 = apolipoprotein E allele 4; BMI = body mass index.
*Nominal p-value, not adjusted for multiplicity; overall p-value and pairwise p-value compared to placebo ≤0.05
^{a}Includes other or missing.

The effect of administration of lanabecestat on weight loss is provided in Table 2 below. Comparing completers at week 104, the mean weight change was for 0.0 kg for placebo (n=202), −0.8 kg for lanabecestat 20 mg (n=187), and −1.9 kg for lanabecestat 50 mg (n=178). The overall p-value and pairwise p-value compared to placebo was ≤0.05 (nominal p-value, not adjusted for multiplicity).

TABLE 2
Effects of Lanabecestat on Weight

| | | | Lanabecestat | | Lanabecestat | | |
|---|---|---|---|---|---|---|---|
| | Placebo | | 20 mg | | 50 mg | | Overall |
| Measurement | N | n (%) | N | n (%) | N | n (%) | p-value |
| Weight | 731 | 93 | 728 | 154 | 723 | 180 | ≤0.05 |
| decrease ≥ 7% | | (12.7) | | (21.2)* | | (24.9)* | |

### Example 2

A Phase 3, multicenter, randomized, double-blind, global study of lanabecestat in patients with mild AD dementia that included a 78-week placebo-controlled period, for which results are provided below.

Lanabecestat was provided orally once daily for 78 weeks at 20 mg or 50 mg and compared to the results for once daily administration of placebo.

The key inclusion criteria for the study were as follows:
Population aged 55 to 85 years;
Mild AD: meet National Institute on Aging and the Alzheimer's Association (NIA-AA) criteria with a Clinical Dementia Rating Scale-Global Score (CDR-GS) of 0.5 or 1, with memory box score ≥0.5;
Mini-Mental State Examination (MMSE) score 20-26;
Study partner;
Amyloid positive
   Cerebrospinal fluid; or
   Florbetapir amyloid Positron Emission Tomography (PET); or
   Historical amyloid PET.

The key exclusion criteria for the study were:
Unstable medical conditions or medications;
Magnetic resonance imaging (MRI): >5 microhemorrhages, significant cerebrovascular pathology, or other pathologies;
QT interval adjusted for heart rate using the Fridericia formula (QTcF) >470 ms;
History of vitiligo and/or current evidence of post-inflammatory hypo-pigmentation.

The baseline demographics for subjects enrolled in the study are summarized in Table 3.

TABLE 3
Baseline Demographics

| | | Lanabecestat | Lanabecestat |
|---|---|---|---|
| | Placebo | 20 mg | 50 mg |
| Demographic | (N = 562) | (N = 590) | (N = 570) |
| Sex, % Female | 61.9% | 56.8% | 59.6% |
| Age, mean | 72.1 | 72.3 | 72.6 |
| Race, % Asian | 11.4% | 12.9% | 12.1% |
| % Black or AA | 0.7% | 0.8% | 1.6% |
| % White | 68.9% | 67.5% | 68.2% |
| % Other^{a} | 19.0% | 18.9% | 18.1% |
| Ethnicity, % Hispanic/Latino | 11.3% | 9.2% | 11.5% |
| Education, % 13 years+ | 54.6% | 54.9% | 55.9% |
| BMI (kg/m²), mean | 25.6 | 25.7 | 25.4 |
| Employment, % full-time | 3.6% | 4.6% | 3.3% |
| APOE 4, % carrier | 71.4% | 66.0% | 65.1%* |
| Family History AD, % 1st | 37.2% | 33.1% | 32.6% |
| Degree, Yes | | | |
| AChEI use, % (n) | 66.2% | 65.9% | 67.5% |

Abbreviations: AA = African American; AChEI = acetylcholinesterase inhibitor; AD = Alzheimer's disease; APOE 4 = apolipoprotein E allele 4; BMI = body mass index.
*Pairwise p-value ≤0.05 (nominal, not adjusted for multiplicity)
^{a}Includes American Indian or Alaskan Native, Native Hawaiian or Other Pacific Islander, multiple, or missing

The effect of administration of lanabecestat on weight loss and on the inhibition of weight gain is provided in Table 4 below. Comparing completers at week 78, the mean weight change was −0.2 kg for placebo (n=24), −1.4 kg for lanabecestat 20 mg (n=27), and −2.9 kg for lanabecestat 50 mg (n=20). The overall p-value and pairwise p-value compared to placebo was ≤0.05 (nominal p-value, not adjusted for multiplicity).

TABLE 4
Effects of Lanabecestat on Weight

| | | | Lanabecestat | | Lanabecestat | | |
|---|---|---|---|---|---|---|---|
| | Placebo | | 20 mg | | 50 mg | | Overall |
| Measurement | N | n (%) | N | n (%) | N | n (%) | p-value |
| Weight | 539 | 34 | 570 | 54 | 545 | 79 | ≤0.05 |
| decrease ≥ 7% | | (6.3) | | (9.5) | | (14.5)* | |

### Example 3

A further analysis of the subjects in the two studies described in Examples 1 and 2 above was performed, for which results are provided below. The baseline demographics for subjects included in the combined analysis are summarized in Tables 5 and 6 below.

TABLE 5
Baseline Demographics

| | | Lanabecestat | Lanabecestat | |
|---|---|---|---|---|
| | Placebo | 20 mg | 50 mg | Total |
| Demographic | (N = 1296) | (N = 1324) | (N = 1303) | (N = 3923) |
| Sex, % Female | 744 (57%) | 725 (55%) | 720 (55%) | 2189 (56%) |
| Age, mean | 71.7 | 71.1 | 71.8 | 71.7 |
| Region, % N America | 455 (35%) | 468 (35%) | 467 (36%) | 1390 (35%) |
| % Europe | 638 (49%) | 628 (47%) | 616 (47%) | 1882 (48%) |
| % Asia | 141 (11%) | 153 (12%) | 162 (12%) | 456 (12%) |
| % ROW | 62 (5%) | 75 (6%) | 58 (4%) | 195 (5%) |
| RACE (White) | 980 (76%) | 1003 (76%) | 976 (75%) | 2959 (75%) |
| Weight (kg), mean (SD) | 69.7 (14.4) | 69.7 (15.0) | 69.5 (15.1) | 69.6 (14.8) |
| BMI (kg/m²), mean (SD) | 25.5 (4.4) | 25.4 (4.3) | 25.3 (4.6) | 25.4 (4.4) |
| <=25 | 668 (52%) | 667 (50%) | 690 (53%) | 2025 (52%) |
| >25 to <=27 | 218 (17%) | 245 (19%) | 234 (18%) | 697 (18%) |
| 27 to <=30 | 227 (18%) | 242 (18%) | 212 (16%) | 681 (17%) |
| >30 | 172 (13%) | 163 (12%) | 165 (13%) | 500 (13%) |

TABLE 6
Baseline Demographics; Weight-related medical history

| Weight-related | | Lanabecestat | Lanabecestat | |
|---|---|---|---|---|
| medical | Placebo | 20 mg | 50 mg | Total |
| history | (N = 1296) | (N = 1324) | (N = 1303) | (N = 3923) |
| Current smoker | 72 (6) | 75 (6) | 64 (5) | 211 (5) |
| Hypertension | 681 (53%) | 650 (49%) | 663 (51%) | 1994 (51%) |
| [a] | | | | |
| Dyslipidaemia | 653 (50%) | 652 (49%) | 651 (50%) | 1956 (50%) |
| [b] | | | | |
| Diabetes | 170 (13%) | 205 (15%) | 193 (15%) | 568 (15%) |
| [c] | | | | |
| Weight Loss | 6 (0.5%) | 10 (0.8%) | 8 (0.6%) | 24 (0.6%) |

[a] Diagnosed at baseline with hypertension.
[b] Diagnosed at baseline with dyslipidaemia, hypercholsterolaemia, hypertriglyceridaemia, hyperlipidaemia, low HDL cholesterol.
[c] Diagnosed at baseline with diabetes.

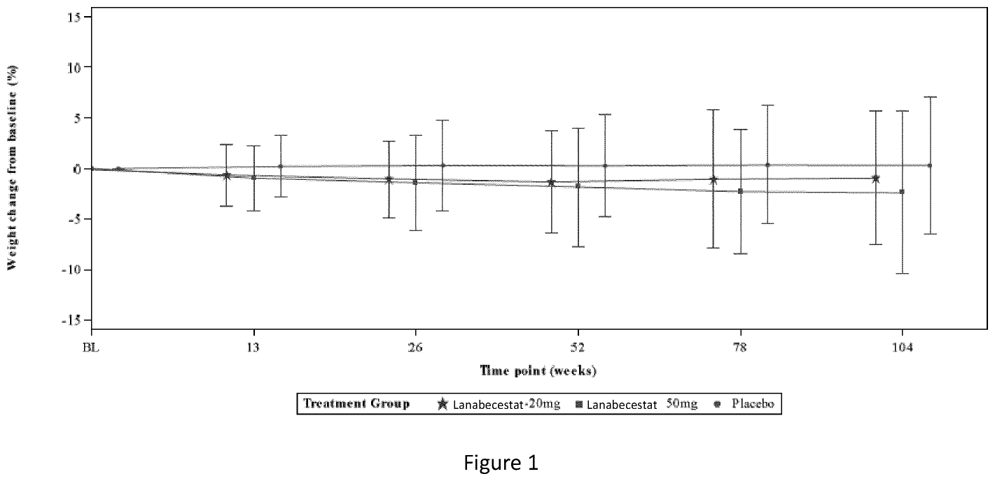

FIGS. 1-3 show weight change from baseline over the course of the 104 week studies. FIG. 1 shows the weight change for the overall population with the N values for the various treatment groups across the various time points provided in Table 7. At 52 weeks, the mean (SD) kg change from baseline in the Lanabecestat 50 mg group versus placebo was −1.5 kg (4.4) vs +0.1 kg (3.4).

TABLE 7
Overall Population Weight Change N Values

| Treatment | Time Point | Time Point | Time Point | Time Point | Time Point |
|---|---|---|---|---|---|
| Group | 13 Weeks | 26 Weeks | 52 Weeks | 78 Weeks | 104 Weeks |
| Lanabecestat | 1228 | 1080 | 726 | 366 | 187 |
| 20 mg (N) | | | | | |
| Lanabecestat | 1194 | 1047 | 716 | 347 | 178 |
| 50 mg (N) | | | | | |
| Placebo (N) | 1202 | 1071 | 743 | 387 | 202 |

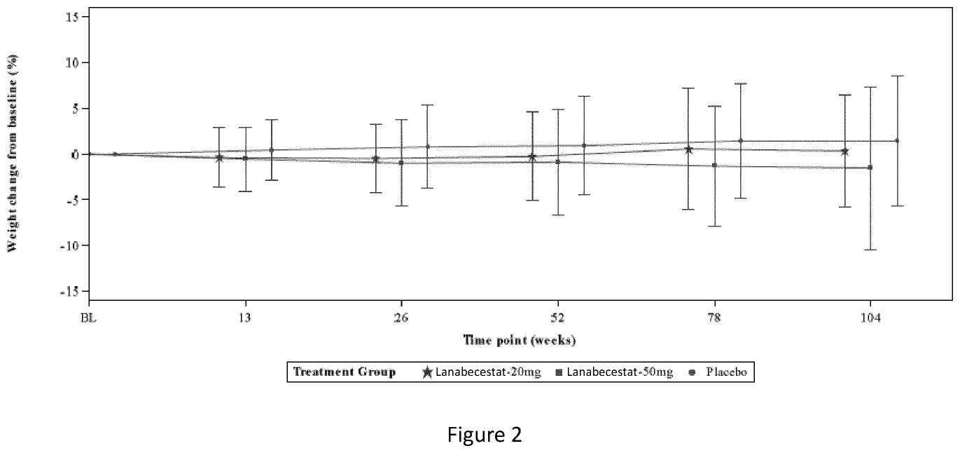

FIG. 2 shows the weight change for the population with a baseline BMI≤25 kg/m² with the N values for the various treatment groups across the various time points provided in Table 8. At 52 weeks, the mean (SD) kg change from baseline in the Lanabecestat 50 mg group versus placebo was −0.65 kg (3.4) vs +0.3 kg (3.3).

TABLE 8
Population With Baseline BMI ≤25 kg/m² Weight Change Demographics

| Treatment | Time Point | Time Point | Time Point | Time Point | Time Point |
|---|---|---|---|---|---|
| Group | 13 Weeks | 26 Weeks | 52 Weeks | 78 Weeks | 104 Weeks |
| Lanabecestat | 619 | 542 | 373 | 193 | 98 |
| 20 mg (N) | | | | | |
| Lanabecestat | 625 | 552 | 367 | 186 | 101 |
| 50 mg (N) | | | | | |
| Placebo (N) | 613 | 551 | 392 | 203 | 106 |

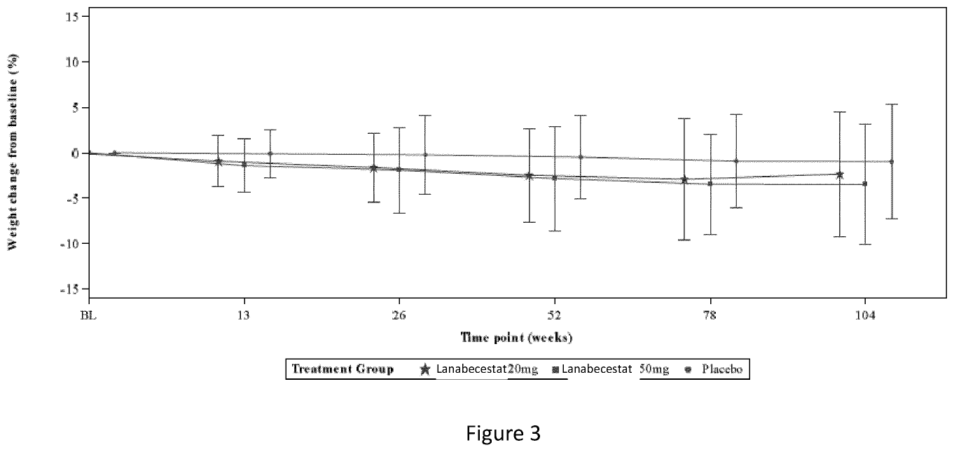

FIG. 3 shows the weight change for the population with a baseline BMI>25 kg/m² with the N values for the various treatment groups across the various time points provided in Table 9. At 52 weeks, the mean (SD) kg change from baseline in the Lanabecestat 50 mg group versus placebo was −2.5 kg (5.2) vs −0.4 kg (3.6), demonstrating that greater weight loss was observed in subjects with a baseline BMI>25 kg/m².

TABLE 9
Population With Baseline BMI >25 kg/m² Weight Change N Values

| Treatment | Time Point | Time Point | Time Point | Time Point | Time Point |
|---|---|---|---|---|---|
| Group | 13 Weeks | 26 Weeks | 52 Weeks | 78 Weeks | 104 Weeks |
| Lanabecestat | 619 | 542 | 373 | 193 | 98 |
| 20 mg (N) | | | | | |
| Lanabecestat | 625 | 552 | 367 | 186 | 101 |
| 50 mg (N) | | | | | |
| Placebo (N) | 613 | 551 | 392 | 203 | 106 |

Table 10 provides data for the weight decrease over time for the population with a baseline BMI>25 kg/m² versus the overall population, showing that there is a greater percent decrease in weight in the higher BMI group versus the overall population. At the one year time point, 30% of the population with a baseline BMI>25 kg/m² demonstrated a weight loss of greater than or equal to 5% as compared to 13% of the placebo group. Treatment with 50 mg Lanabecestat has shown a statistically significant weight loss of greater than or equal to 5% and greater than or equal to 10% at 2 years.

TABLE 10
Weight (kg) decrease over time

| | % | | | | | | |
|---|---|---|---|---|---|---|---|
| Time | Weight | All patients | | | Patients with baseline BMI > 25 | | |
| (week) | Loss | Pcbo | L20 | L50 | Pcbo | L20 | L50 |
| 26 | N | 1071 | 1080 | 1047 | 512 | 532 | 494 |
| | ≥5% | 72 (7%) | 147 | 172 | 38 (7%) | 87 | 90 |
| | | | (14%)*** | (16%)*** | | (16%)*** | (18%)*** |
| | ≥10% | 10 (1%) | 15 (1%) | 28 | 6 (1%) | 11 (2%) | 20 (4%)** |
| | | | | (3%)** | | | |
| | ≥15% | 3 (0.3%) | 3 (0.3%) | 5 (0.5%) | 2 (0.4%) | 3 (0.6%) | 3 (1%) |
| 52 | N | 743 | 726 | 716 | 346 | 348 | 348 |
| | ≥5% | 88 (12%) | 139 | 180 | 44 (13%) | 82 | 104 |
| | | | (19%)*** | (25%)*** | | (24%)*** | (30%)*** |
| | ≥10% | 17 (2%) | 35 (5%)* | 39 | 13 (4%) | 26 (7%)* | 26 (7%)* |
| | | | | (5%)** | | | |
| | ≥15% | 4 (0.5%) | 8 (1%) | 8 (1%) | 4 (1%) | 8 (2%) | 6 (2%) |
| 78 | N | 387 | 366 | 347 | 181 | 170 | 161 |
| | ≥5% | 58 (15%) | 85 | 107 | 35 (19%) | 52 | 57 |
| | | | (23%)** | (31%)*** | | (31%)* | (35%)** |
| | ≥10% | 14 (4%) | 26 (7%)* | 36 | 9 (5%) | 17 (10%) | 20 (12%)* |
| | | | | (10%)*** | | | |
| | ≥15% | 3 (1%) | 9 (2%) | 7 (2%) | 2 (1%) | 9 (5%)* | 5 (3%) |
| 104 | N | 202 | 187 | 178 | 95 | 89 | 77 |
| | ≥5% | 39 (19%) | 48 (26%) | 67 | 20 (21%) | 27 (30%) | 32 |
| | | | | (38%)*** | | | (42%)** |
| | ≥10% | 12 (6%) | 16 (9%) | 31 | 8 (8%) | 13 (15%) | 1 5 (19%)* |
| | | | | (17%)*** | | | |
| | ≥15% | 1 (0.5%) | 4 (2%) | 6 (3%) | 1 (1%) | 4 (4%) | 3 (4%) |

Pairwise p-value vs placebo via Fisher's exact test: *p < 0.05, **p < 0.01, ***p < 0.001

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject application have been discussed, the above specification is illustrative and not restrictive. Many variations of the subject of the application will become apparent to those skilled in the art upon review of this specification and the claims below. The full scope of the application should be determined by reference to the claims, along with their full scope of equivalents, and the specification, along with such variations.

1. A method of affecting weight loss in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, 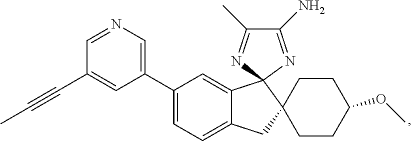 [SMILES: CC#Cc1cncc(-c2ccc3c(c2)[C@@]2(N=C(C)C(N)=N2)[C@]2(CC[C@H](OC)CC2)C3)c1]
or a pharmaceutically acceptable salt thereof.

2. The method of claim 1, wherein the weight of the subject decreases by at least 5%, at least 6%, at least 7%, at least 8%, at least 9%, at least 10%, at least 15%, at least 20%, or at least 30%.

3. The method of claim 1 or 2, wherein the weight loss is maintained for at least one month.

4. The method of claim 1 or 2, wherein the weight loss is maintained for at least one year.

5. A method of preventing weight gain in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, 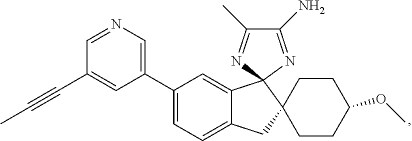 [SMILES: CC#Cc1cncc(-c2ccc3c(c2)[C@@]2(N=C(C)C(N)=N2)[C@]2(CC[C@H](OC)CC2)C3)c1]
or a pharmaceutically acceptable salt thereof.

6. The method of claim 5, wherein the weight of the subject increases by less than 5%.

7. The method of claim 5 or 6, wherein the prevention of weight gain is maintained for at least one month.

8. The method of claim 5 or 6, wherein the prevention of weight gain is maintained for at least one year.

9. A method of reducing body mass index (BMI) in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, 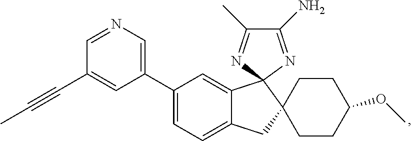 [SMILES: CC#Cc1cncc(-c2ccc3c(c2)[C@@]2(N=C(C)C(N)=N2)[C@]2(CC[C@H](OC)CC2)C3)c1]
or a pharmaceutically acceptable salt thereof.

10. The method of claim 9, wherein the subject has a BMI of at least about 25 kg/m2 prior to the administration.

11. The method of claim 9 or 10, wherein the BMI is reduced by at least 5%, at least 6%, at least 7%, at least 8%, at least 9%, at least 10%, at least 15%, at least 20%, or at least 30%.

12. The method of any one of claims 9-11, wherein the subject has a BMI of less than 25 kg/m2 after the administration of the therapeutically effective amount.

13. The method of any one of claims 9-12, wherein the reduced BMI is maintained for at least one month.

14. The method of any one of claims 9-12, wherein the reduced BMI is maintained for at least one year.

15. A method of treating a condition or disorder associated with being overweight or obese in a subject comprising administration of a therapeutically effective amount of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, 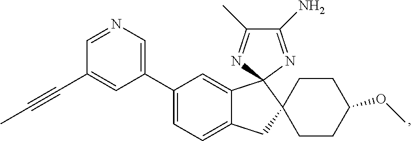 [SMILES: CC#Cc1cncc(-c2ccc3c(c2)[C@@]2(N=C(C)C(N)=N2)[C@]2(CC[C@H](OC)CC2)C3)c1]
or a pharmaceutically acceptable salt thereof.

16. The method of claim 15, wherein the condition or disorder associated with being overweight or obese comprises a cardiac disorder, an endocrine disorder, a respiratory disorder, a hepatic disorder, a skeletal, a psychiatric disorder, a metabolic disorder, a sleeping disorder, a reproductive disorder, or a combination thereof.

17. The method of any preceding claim, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, 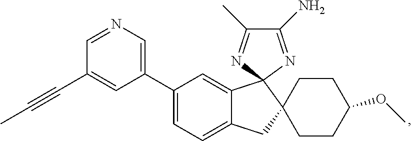 [SMILES: CC#Cc1cncc(-c2ccc3c(c2)[C@@]2(N=C(C)C(N)=N2)[C@]2(CC[C@H](OC)CC2)C3)c1]
or a pharmaceutically acceptable salt thereof, is conjointly administered with a second weight loss agent.

18. The method of claim 17, wherein the second weight loss agent is selected from serotonin and noradrenergic re-uptake inhibitors, noradrenergic re-uptake inhibitors, selective serotonin re-uptake inhibitors, intestinal lipase inhibitors, orlistat, sibutramine, methamphetamine, ionamin, phentermine, bupropion, diethylpropion, phendimetrazine, benzphetermine, pramlintide, exenatide, liraglutide, and Topamax.

19. The method of any preceding claim, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered orally.

20. The method of any preceding claim, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered once daily.

21. The method of claim 20, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered at a dose of 20 mg/day.

22. The method of claim 20, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered at a dose of 50 mg/day.

23. The method of any preceding claim, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered once daily for at least 5 weeks.

24. The method of any preceding claim, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered once daily for at least 25 weeks.

25. The method of any preceding claim, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is administered for at least 50 weeks.

26. The method of any preceding claim, wherein the (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, is the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine,

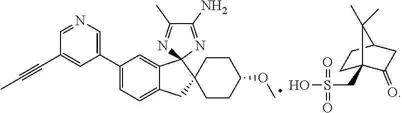 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

27. Use of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, in the preparation of a medicament for affecting weight loss.
